(19) United States
(12) Patent Application Publication
(10) Pub. No.: US 2013/0210710 A1
(43) Pub. Date: Aug. 15, 2013

(54) TREATMENT OF HIV

(71) Applicant: AIMSCO LIMITED, (US)

(72) Inventor: Deirdre McIntosh, Sussex (GB)

(73) Assignee: AIMSCO LIMITED, East Sussex (GB)

(21) Appl. No.: 13/693,174

(22) Filed: Dec. 4, 2012

Related U.S. Application Data
(63) Continuation of application No. 13/450,597, filed on Apr. 19, 2012, now abandoned.
Application No. 13/450,597 is a continuation of application No. 12/087,442, filed on Dec. 11, 2008, now abandoned.

(30) Foreign Application Priority Data
Jan. 6, 2006 (GB) 00600202.6

(51) Int. Cl. A61K 38/22; A61K 38/35

(52) U.S. Cl. CPC A61K 38/2228; A61K 38/35; A61K 38/22
USPC 514/3.9; 514/3.8

(57) ABSTRACT

We describe methods of treatment of HIV using proopiomelanocortin (POMC) and corticotropin releasing factor (CRF) peptides and their products, as well as uses of such peptides in the preparation of medicaments.

### FIELD OF THE INVENTION

The present invention relates to methods of treatment of HIV, and to use of POMC and/or CRF peptides in the preparation of medicaments for the treatment of HIV.

### BACKGROUND TO THE INVENTION

The human immunodeficiency virus/acquired immunodeficiency syndrome (HIV/AIDS) epidemic has caused over 20 million deaths worldwide and currently affects about 40 million people. This has a serious socio-economic impact particularly on developing countries. To date, the only effective weapon against HIV and AIDS is therapy, notably highly active anti-retroviral therapy (HAART). However, this is not available worldwide, can have toxic side effects and often those most in need are deprived of treatment. Therefore the requirement for an effective therapeutic HIV vaccine or prophylactic treatment has become increasingly extremely urgent.

International Patent Application PCT/GB2005/050108 describes the use of corticotropin releasing factor (CRF) and/or proopiomelanocortin (POMC) peptides in the treatment of a range of disorders in patients. The reader is referred to PCT/GB2005/050108 for a list of the disorders which may be treated. Preparation of a goat serum product is described in International Patent applications WO03/004049 and WO03/064472; we now believe that this serum product may be a useful source of CRF and POMC peptides which may be used in the present invention.

We have now discovered that CRF and/or POMC peptides are useful in the treatment of HIV, and in particular in the reduction of viral load and/or increase in CD4+ and CD8 cell counts in patients.

CRF is a peptide produced in the hypothalamus, and is believed to be involved in stress response. Human CRF is described in detail in entry 122560 of OMIM (online mendelian inheritance in man, accessible through http://www.ncbi.nlm.nih.gov/). The nucleotide and amino acid sequence of human CRF is also known, and has GENBANK accession number BC011031. Knowledge of the sequence and size data for human CRF will allow the skilled person to determine the equivalent information for non-human CRF, including goat CRF. CRF is also known as corticotropin releasing hormone (CRH).

By “a CRF peptide” is meant any peptide having a corresponding sequence, structure, or function. It will be apparent to the skilled person that the canonical nucleotide and/or amino acid sequences given for human CRF in the GENBANK entry referenced above may be varied to a certain degree without affecting the structure or function of the peptide. In particular, allelic variants and functional mutants are included within this definition. Mutants may include conservative amino acid substitutions; and fragments and derivatives of CRF.

Administration of CRF to a patient is believed to stimulate production of endogenous CRF, which in turn stimulates production of proopiomelanocortin (POMC) and its related component peptides.

POMC is a peptide (prohormone) produced in the pituitary gland (as well as a number of other organs, certain tumours such as melanomas, and normal skin cells) which is the precursor of a set of corticotrophic hormones which exert a number of effects on the host. POMC is the precursor to alpha, beta, and gamma melanocyte stimulating hormone (MSH); adrenocorticotrophin (ACTH); beta and gamma lipotropin (LPH); and beta endorphin. All of these hormones are cleaved from a single large precursor, POMC, and are termed herein “POMC products”.

Human POMC is described in detail in entry 176830 of OMIM (online mendelian inheritance in man, accessible through http://www.ncbi.nlm.nih.gov/). The nucleotide and amino acid sequence of human POMC is also known, and has GENBANK accession number BC065832. Human POMC gives rise to a glycosylated protein precursor having a molecular weight of 31 kDa.

By “a POMC peptide” is meant any peptide having a corresponding sequence, structure, or function. It will be apparent to the skilled person that the canonical nucleotide and/or amino acid sequences given for human POMC in the GENBANK entry referenced above may be varied to a certain degree without affecting the structure or function of the peptide. In particular, allelic variants and functional mutants are included within this definition. Mutants may include conservative amino acid substitutions. “A POMC peptide” as used herein refers to any peptide acting as a precursor to at least one form of MSH, ACTH, at least one form of LPH, β endorphin, met-enkephalin and leu-enkephalin; and preferably all of α, β, and γ MSH; ACTH; β and γ LPH; and β endorphin, met-enkephalin and leu-enkephalin

### SUMMARY OF THE INVENTION

According to a first aspect of the present invention, there is provided a method of treatment of HIV comprising administering a corticotropin releasing factor (CRF) peptide to a patient.

The treatment may be used to obtain one or more of the following effects: a reduction in viral load; an increase in CD4 cells; or an increase in CD8 cells. We believe that the treatment can be successfully used against HIV and AIDS in human patients. Without wishing to be bound by theory, we believe that the treatment limits and controls virus spread in the body by reducing the levels of the hyperactive immune response necessary for virus replication and spread. In addition, it may control inflammation elicited by opportunistic infections and the consequent production of pro-inflammatory cytokines that support and stimulate viral replication and spread. As such it reduces the viral load in HIV patients, increases the CD4 and CD8 counts in the blood, improves libido, stimulates appetite and improves significantly the quality of life of HIV/AIDS patients.

The CRF may be non-human CRF; conveniently ungulate CRF; and most preferably goat CRF. It has been surprisingly identified that goat serum contains CRF, particularly when the goat is stimulated by physiological stress, such as bleeding or immunization. This provides a convenient source for CRF for pharmaceutical compositions of the present invention. It is also believed that CRF may have a self-sustaining effect in the patient, in that administration of an initial amount of CRF leads to endogenous production of CRF in the patient; thus, an initial administration of a low level of CRF may have a significant effect on the patient, including an increase in the levels of POMC peptides. Of course, peptides obtained from animals other than goats may be used, as may recombinant or other sources of peptide.

Administration of peptides as used in the present invention may be accomplished orally or parenterally. Methods of parenteral delivery include topical, intra-arterial, intramuscular, subcutaneous, intramedullary, intrathecal, intraventricular, intravenous, intraperitoneal, or intranasal administration. In addition to the active ingredients, administered compositions may comprise suitable pharmaceutically acceptable carriers comprising excipients and other components which facilitate processing of the active compounds into preparations suitable for pharmaceutical administration.

Pharmaceutical compositions for oral administration can be formulated using pharmaceutically acceptable carriers known in the art in dosages suitable for oral administration. Such carriers enable the compositions to be formulated as tablets, pills, dragees, capsules, liquids, gels, syrups, slurries, suspensions, and the like suitable for ingestion by the subject.

Pharmaceutical preparations for oral use can be obtained through combination of active compounds with a solid excipient, optionally grinding a resulting mixture, and processing the mixture of granules, after adding suitable additional compounds if desired to obtain tablets or dragee cores. Suitable excipients include carbohydrate or protein fillers such as sugars, including lactose, sucrose, mannitol, sorbitol; starch from corn, wheat, rice, potato, or other plants; cellulose such as methylcellulose, hydroxypropylmethylcellulose, or sodium carboxymethylcellulose; and gums including arabic and tragacanth; as well as proteins such as gelatin and collagen. If desired, disintegrating or solubilising agents may be added, such as cross linked polyvinyl pyrrolidone, agar, alginic acid, or a salt thereof.

Dragee cores can be provided with suitable coatings such as concentrated sugar solutions, which may also contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for product identification or to characterise the quantity of active compound.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally stabilisers. In soft capsules, the active compounds can be dissolved or suspended in suitable liquids, such as fatty oils, liquid paraffin, or liquid polyethylene glycol with or without stabilisers.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous suspension injections can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilisers or agents which increase the solubility of the compounds to allow for the preparation of highly concentrated solutions.

For topical or nasal administration, penetrants appropriate to the particular barrier to be permeated may be used in the formulation.

Pharmaceutical compositions for use in the present invention can be manufactured substantially in accordance with standard manufacturing procedures known in the art.

Peptides or compositions for use in the present invention may be lyophilised. This improves storage life and stability of the product, and improves transportability. This is particularly beneficial for use in warm climates, and where refrigeration facilities may not be readily available. Lyophilised product may be reconstituted before administration.

The method may further comprise administering one or more peptide regulatory or releasing factors, which may induce a cascade of release of further peptides by a variety of cells in the patient. Such additional factors are preferably derived from the same source as the CRF, in particular goat serum. Suitable factors include α-HLA, TGF-β, and IL-10, among others.

In preferred embodiments, the method may comprise administering one or more of vasopressin, beta endorphin, and an enkephalin. In certain embodiments, the method may comprise administering CRF binding protein, CRF-BP. This binds CRF and may act as a reservoir for subsequent release of CRF to the patient.

The method may further comprise administering a POMC peptide or a POMC product; certain POMC products may be useful to administer to a patient to stimulate further production, or to obtain a desired response before endogenous POMC can be produced.

A further aspect of the present invention provides a method of treatment of HIV comprising administering a POMC peptide and/or a POMC product to a patient.

Preferably the POMC is non-human POMC; conveniently ungulate POMC; and most preferably goat POMC. Although POMC is produced in the pituitary gland, and so would not be expected to be present in serum, at least at significant levels, it has been surprisingly identified that goat serum contains POMC, POMC-related peptides, and molecules associated with the POMC cascade, particularly when the goat is stimulated by physiological stress, such as bleeding or immunization. This provides a convenient source for POMC for pharmaceutical compositions of the present invention. It is also believed that POMC may have a self-sustaining effect in the patient, in that administration of an initial amount of POMC leads to endogenous production of POMC in the patient; thus, an initial administration of a low level of POMC may have a significant effect on the patient. As with CRF peptides, sources of POMC peptides other than goat may of course be used, including recombinant POMC.

It is believed that, on administration of POMC and its associated molecules to a subject, the peptide is proteolysed to provide one or more of the products of POMC in a readily available form to the subject; there is also the induction of a molecular cascade which stimulates the hypothalamo-pituitary-adrenal axis (HPA).

According to a further aspect of the invention, there is provided a method of treatment of HIV comprising administering two or more of alpha, beta, and gamma melanocyte stimulating hormone (MSH); adrenocorticotrophin (ACTH); beta and gamma lipotropin (LPH); and beta endorphin. Given the likely proteolysis of POMC on administration, it may be possible to achieve similar effects by administration of two or more of the individual hormones derived from POMC. The recited hormones may be provided as individual peptides, or as one or more precursor molecules (for example, partial breakdown products of POMC). Preferably three, four, five, six, or seven of the hormones are included in the pharmaceutical composition which (optionally together with CRF) induce a cascade for continued production of such molecules. The various components may be provided in combination with one or more carrier molecules which bind one or more of the components, and so act as a depot or reservoir for release of the component. A carrier molecule may also be used in combination with POMC and its related peptides.

The optimal dosage of POMC or CRF peptides has not yet been determined; however it may be appropriate to administer the peptides in a dosage of between 0.01 and 10 mg/kg to the subject; more preferably between 0.01 and 5 mg/kg, between 0.025 and 2 mg/kg, and most preferably between 0.05 and 1 mg/kg.

The precise dosage to be administered may be varied depending on such factors as the age, sex and weight of the patient, the method and formulation of administration, as well as the nature and severity of the disorder to be treated. Other factors such as diet, time of administration, condition of the patient, drug combinations, and reaction sensitivity may be taken into account. An effective treatment regimen may be determined by the clinician responsible for the treatment. One or more administrations may be given, and typically the benefits are observed after a series of at least three, five, or more administrations. Repeated administration may be desirable to maintain the beneficial effects of the composition.

The treatment may be administered by any effective route, preferably by subcutaneous injection, although alternative routes which may be used include intramuscular or intralesional injection, oral, aerosol, parenteral, or topical.

The treatment is preferably administered as a liquid formulation, although other formulations may be used. The liquid formulation may be reconstituted from a lyophilised preparation. For example, the treatment may be mixed with suitable pharmaceutically acceptable carriers, and may be formulated as solids (tablets, pills, capsules, granules, etc) in a suitable composition for oral, topical or parenteral administration.

The invention also provides the use of CRF in the preparation of a medicament for the treatment of HIV. Also provided is the use of POMC in the preparation of a medicament for the treatment of HIV. The CRF or the POMC may be isolated, purified CRF or POMC, although it is preferred that they are administered in combination with the various other components as discussed above. In particular, bioactive carrier proteins and vasopressin may be used.

### BRIEF DESCRIPTION OF THE DRAWINGS

These and other aspects of the present invention will now be described by way of example only with reference to the accompanying drawings, in which:

FIGS. 1 to 4 show mass spectrometry analyses of tryptic digests of serum components; and

FIGS. 5 to 9 show evidence for a switch in inflammatory profile of patients following treatment with the composition.

### DETAILED DESCRIPTION OF THE INVENTION

International patent publications WO03/004049 and WO03/064472 describe the production of a goat serum composition. A summary of the production method is given below.

### Preparation of Serum Composition

A goat is inoculated by intramuscular injection with lysed HIV-3b virus suspended in a normal commercial supernate, using an intra-muscular injection of HIV-3b at a concentration of 10⁹ viral particles per ml. The virus is previously heat killed at 60° C. for 30 minutes. In the optimised procedure, the goat is injected every week for four weeks, then at six weeks the animal is bled to obtain the reagent.

Approximately 400 cc of blood is taken from a goat under sterile technique. The animal may typically be re-bled in 10 to 14 days, once the volume of blood is replenished. A pre-bleeding regime may be useful to stimulate production of the active components of the serum. All subsequent preparation steps are preferably carried out at 4° C., unless otherwise specified. The blood is then centrifuged to separate the serum, and the serum filtered to remove large clots and particulate matter. The serum is then treated with supersaturated ammonium sulphate (47% solution at 4° C.) to precipitate antibodies and other material. The resulting solution is centrifuged in a Beckman J6M/E centrifuge at 3500 rpm for 45 minutes, after which the supernatant fluid is removed. The precipitated immunoglobulin and other solid material are resuspended in PBS buffer (phosphate buffered saline) sufficient to redissolve the precipitate.

The solution is then subjected to diafiltration against a PBS buffer with a molecular weight cut-off of 10,000 Daltons at 4° C. After diafiltration the product is filtered through a 0.2 micron filter into a sterile container and adjusted to a protein concentration of 4 to 5 mg/ml. The solution is put into vials to give single doses of 1 ml, and stored at −22° C. prior to use.

### Analysis of Serum Composition

PCT/GB2005/050108 reports that serum composition prepared in this way contains POMC and CRF peptides, and suggests an active role for these peptides in the effects of the serum. A summary of the analysis of the serum is given below.

A sample of the composition was size fractionated on a gel, and a Western blot performed using antibodies to β endorphin. A strong signal was detected, indicating the presence of β endorphin, although the apparent molecular weight was approximately 31 kDa, far larger than the expected size of β endorphin. This suggested that β endorphin was present in the sample as part of a larger peptide; the size being consistent with that of POMC.

We have also carried out mass spectrometry on the composition, and have detected at least two POMC-derived peptides, β endorphin and corticotrophin-related molecules. CRH-BP (corticotropin releasing hormone binding protein) has also been identified.

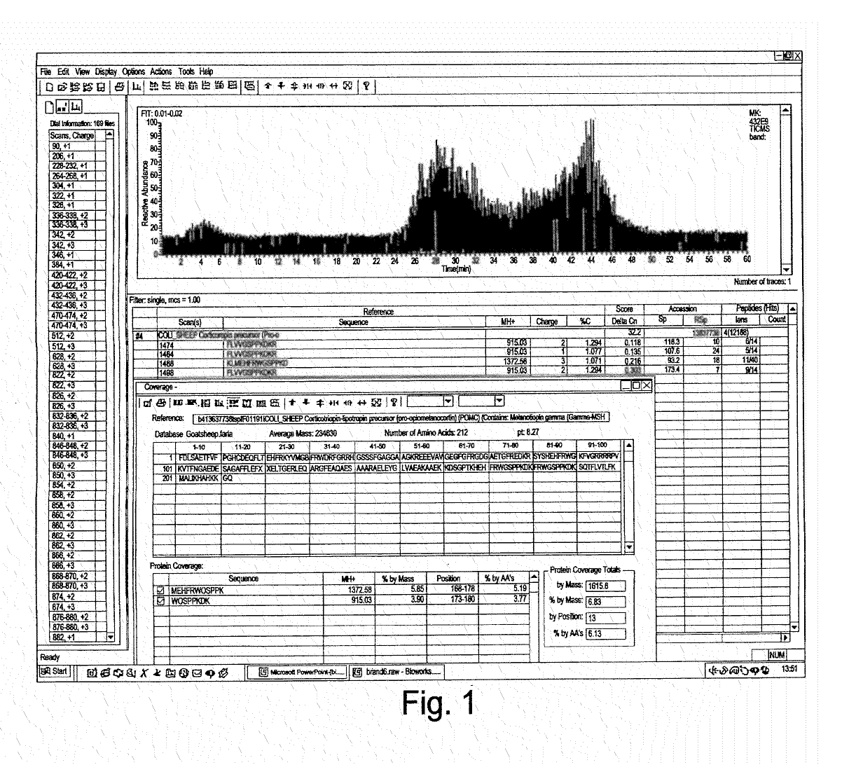

### FIGS. 1 to 4

POMC peptides and CRF-BP have been identified in the product by Thermofinnegan LCQ mass spectrometry. CRF mainly regulates the synthesis and secretion of ACTH in the anterior pituitary. The administration of POMC and/or its component peptides in addition to CRF and CRF-BP is thought to initiate a cascade effect thus enhancing the production of systemic and sustained elevated concentrations of POMC peptides. CRF-BP has the ability to act as a reservoir for CRF.

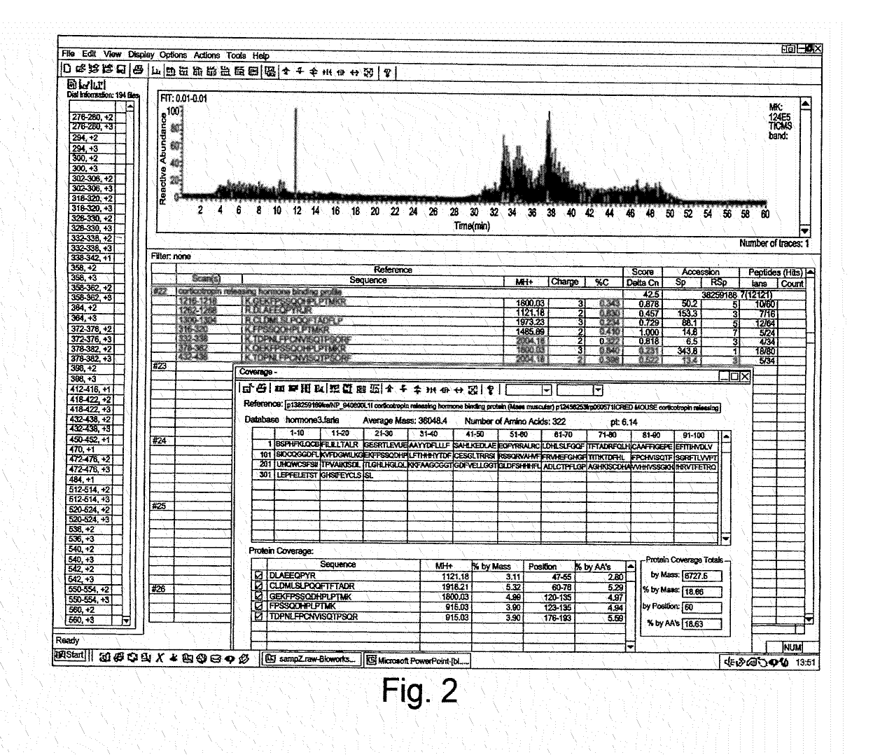

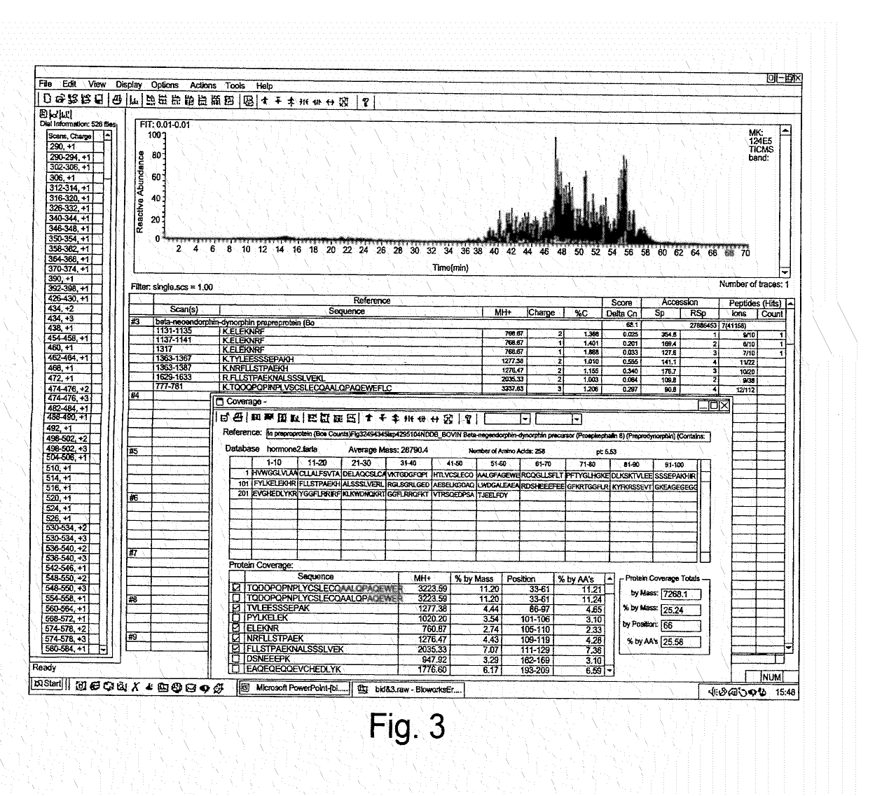

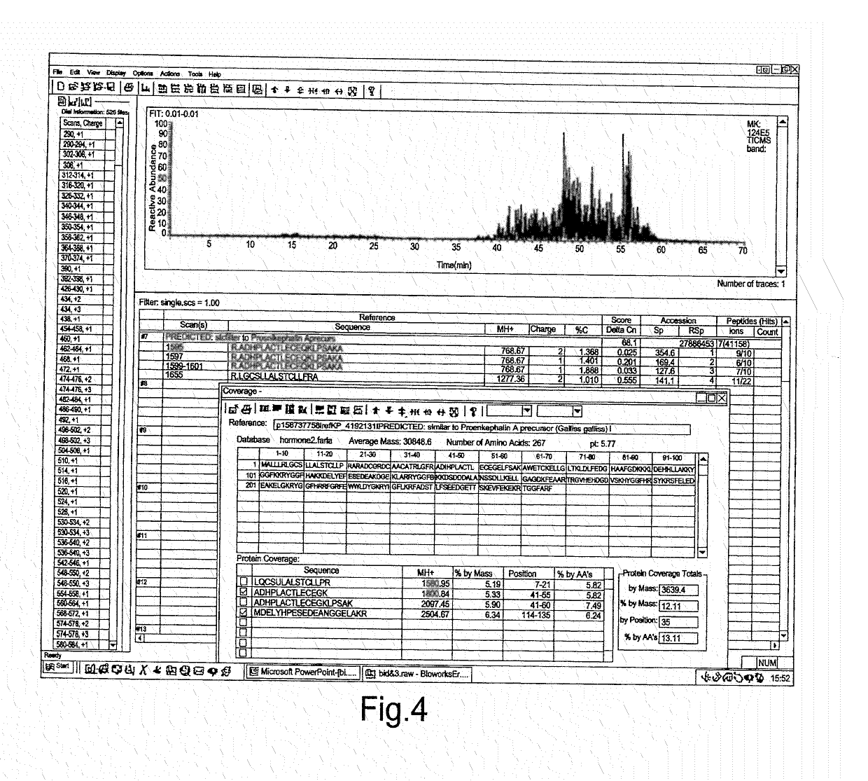

FIGS. 1 to 4 show the hits obtained from mass spectrometry analysis of tryptic digests from the product separated from contaminating proteins by SDS-PAGE. As mentioned above, some of these molecules are inducers and regulators of the POMC cascade. Further investigation using more focused analysis (e.g. peptide fractionation, immunoprecipitation and concentration) will reveal more of the peptides present. FIG. 1 indicates the presence of a POMC-derived corticotropin, FIG. 2 that of CRF-BP, FIG. 3 that of proenkephalin A, and FIG. 4 that of proenkephalin B. The presence of CRF-BP suggests that the product contains some CRF, while POMC and related peptides are also clearly present.

We have also investigated the effects of treatment with the serum composition on patients' own sera. These effects are described below.

Evidence for a Switch from a Pro-Inflammatory TH-1 Profile to an Anti-Inflammatory TH-2 Cytokine Profile in Treated Patients

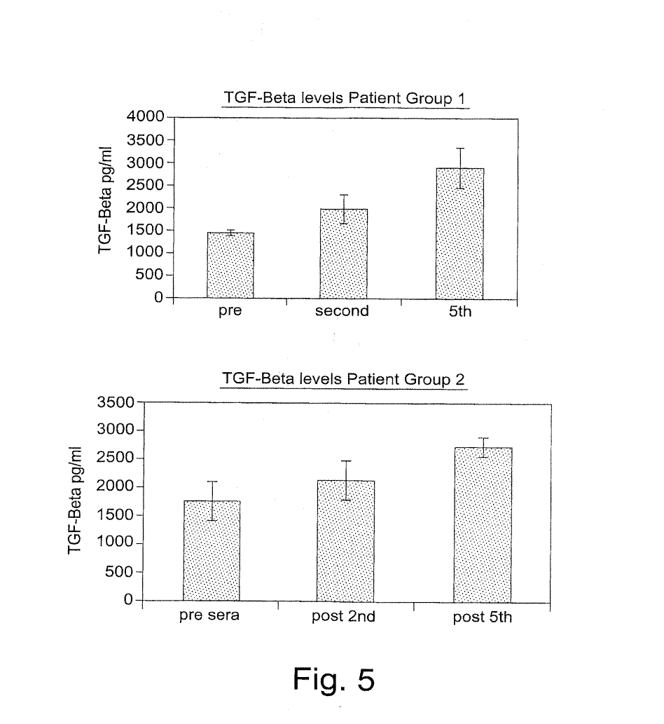

FIG. 5 shows the levels of TGF-β in the serum of two groups of patients (healthy volunteers) before and after treatment with goat serum product prepared as described. The two groups of patients (n=3 for each group) show differing responses with respect to the concentrations of TGF-β produced, but all patients showed an increase in serum levels in response to treatment (pre sera=patients' serum levels before treatment; post 2^{nd} and post 5^{th}=after the 2^{nd} and 5^{th} administration). The data show that treatment induces increased concentration of the anti-inflammatory cytokine TGF-β.

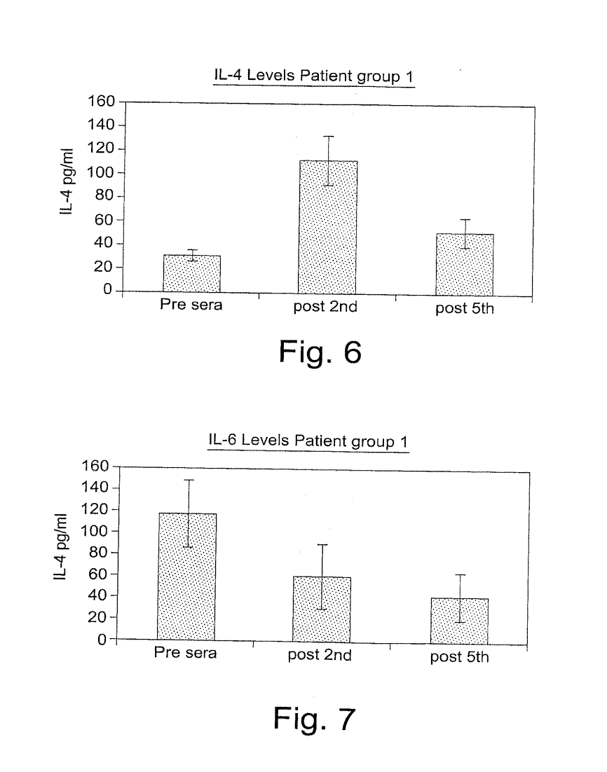

FIG. 6 shows the levels of IL-4 in the serum of one group of patients before (pre-sera) and after treatment. It can be seen that after treatment (post 2^{nd}), the levels of IL-4 are significantly increased in the patients' sera (n=5). However, following the 5^{th} administration, the levels of IL-4 had dropped in all patients, but remained higher than they had been pre-treatment. IL-4 is known to downregulate the production of the pro-inflammatory cytokines from TH-1 cells. It may be that the consistent changes in concentration seen in all patients is consistent with IL-4's role in the TH-1 to TH-2 switch.

FIG. 7 shows the levels of IL-6 in the serum of one group of patients before and after treatment. It can be seen that after treatment (post 2^{nd} and post 5^{th}) the levels of IL-6 are reduced in the patients' sera (n=4).

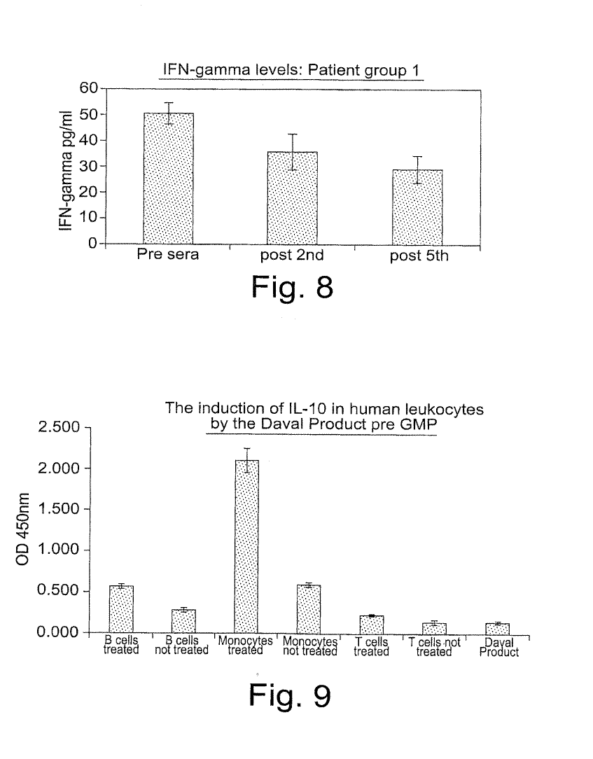

FIG. 8 shows the levels of IFN-γ in the serum of one group of patients before and after treatment. It can be seen that after treatment (post 2^{nd} and post 5^{th}) the levels of IFN-γ are reduced in the patients' sera.

FIG. 9 shows that treatment of human peripheral blood cells (PBMCs) induces the production of the anti-inflammatory cytokine IL-10 in the monocyte sub population. T and B lymphocytes and monocytes were separated from PBMCs obtained from human volunteers. All cell types were treated with equivalent doses of product for 16 h, and their supernatants assayed for IL-10 content using ELISA. It can be seen that IL-10 levels produced by the T cell population were unaffected by treatment and that only a small increase in IL-10 was induced in the B cells. However, a significant elevation of IL-10 concentration was induced in the monocytes population by the treatment. All determinations were made in triplicate +/−standard deviations. These data are representative of at least three separate experiments.

### SUMMARY AND CONCLUSIONS

We show above and in PCT/GB2005/050108 that the goat serum product as described contains POMC peptides and products, and CRF peptides. We also show that administration of the serum product induces a switch in the inflammatory profile of patients.

WO03/004049 describes the use of goat serum product prepared as described for the treatment of patients with HIV. It is suggested in that publication that the beneficial results of the serum on HIV result from the presence of anti-FAS and anti-HLA molecules; there Is no suggestion that POMC or CRF peptides may be present. The publication observes that patients given the serum experience an increase in CD4 and CD8 cell count, reduction in viral load, and reduction of P24 values.

WO02/07760 also describes the preparation and use of the same goat serum product to treat patients with HIV. The publication reports experimental data showing the neutralisation of SIV in vitro. Example 3 of the publication describes the preparation of goat serum product in the same manner as described above. Administration of the serum results in a decrease In HIV viral load (defined as the number of copies of HIV-1 RNA per ml of plasma), and an increase in CD4 and CD8 cell count. Again, no suggestion that these properties may result from the presence of POMC or CRF peptides is made.

In view of the findings of PCT/GB2005/050108, and the data presented herein, that the goat serum product contains POMC peptides and products, and CRF peptides, and that these peptides and products are active biological agents, we believe that these peptides and products may be useful in the treatment of HIV and/or AIDS in human patients, to obtain among other effects one or more of a reduction in viral load, increase in CD4 cell count, and an increase in CD8 cell count.

HIV is also known to induce a variety of lesions of the central nervous system (CNS) which lead to neurodegeneration and a range of neuropathologies. These include HIV encephalitis, HIV leukoencephalopathy, axonal damage, and diffuse poliodystrophy, which is associated with neuronal loss of variable severity. The latter is thought to result from an apoptotic process. These conditions result in loss of cognitive function and dementia. In view of the described effects of POMC/CRF peptides on neurodegenerative disorders (see PCT/G2005/050108), it is likely that such peptides may be used to alleviate these symptoms of HIV/AIDS as well as HIV/AIDS itself.

It has also been reported that the hypothalamo pituitary adrenal (HPA) axis in people infected with the HIV virus is dysfunctional. The manipulation of the cytokine network could have beneficial effects in the control of HIV infection. The stimulation of melanocortin receptors on inflammatory cells might be an effective therapeutic approach to alter the course of HIV infection. Proopiomelanocortin-derived peptides present in the serum product described herein include adrenocorticotropic hormone [ACTH (1-39)], α-melanocyte-stimulating hormone [α-MSH (1-13)], and related amino acid sequences. Melanocortin peptides have potent antiinflammatory/anticytokine activity.

Cytokines such as interleukin 1 (IL-1) and tumor necrosis factor (TNF) can be detrimental in HIV-infected patients. The effects of melanocortins on the production of IL-1 and TNF-α in the blood of HIV patients have been investigated. When cytokine production was measured in whole blood samples stimulated with LPS in the presence or absence of α-MSH (1-13), α-MSH (11-13), ACTH (1-24), or ACTH (1-39) it was found that melanocortins reduced the production of both cytokines in a concentration-dependent manner. In separate experiments on normal peripheral blood mononuclear cells (PBMC), α-MSH (1-13) was found to inhibit the production of IL-1β and TNF-α induced by HIV envelope glycoprotein gp 120. These results suggest that stimulation of melanocortin receptors in inflammatory cells could be a novel way to reduce production of cytokines that promote HIV replication.

POMC and CRF peptides and products, either individually or in combination, provide a novel pharmaceutical product that has the capacity to regulate the HPA axis and serve as a source of melanocortins and regulator of melanocortin production. In particular it appears to convert a hyperactive pro-inflammatory TH1 cytokine into an anti-inflammatory TH2 profile. Thus the production and release of inflammatory cytokines is regulated.

Evidence exists that supports the notion that HIV infection is facilitated by the infection of monocyte-macrophages by multiple pathways. The activation of NF-kB by opportunistic infections in AIDS augments the expression of CCR5 receptors and the expression of TNF-α, both of which are permissive for sustaining HIV infections. In additionally, it has been found that a reduction in viral burden is associated with the treatment of infected and/or inflamed tissue; this further supports the link between immune activation and viral replication. Thus, treatment of patients with POMC/CRF peptides and/or products, which reduce tissue inflammation and levels of level of pro-inflammatory cytokines and macrophage activation will reduce cellular infectivity in the patient and possibly the spread of the infection to body organs such as the brain.

1-13. (canceled)

14. A method of treatment of HIV comprising administering a corticotropin releasing factor (CRF) peptide to a patient.

15. The method of claim 14, wherein one or more of the following effects is achieved: a reduction in viral load; an increase in CD4 cells; or an increase in CD8 cells.

16. The method of claim 14 wherein the CRF is non-human CRF.

17. The method of claim 16 wherein the CRF is goat CRF.

18. The method of claim 14, and further comprising administering a POMC peptide or a POMC product.

19. The method of claim 18, and further comprising administering two or more of alpha, beta, and gamma melanocyte stimulating hormone (MSH); adrenocorticotrophin (ACTH); beta and gamma lipotropin (LPH); and beta endorphin.
